(19) United States
(12) Patent Application Publication
(10) Pub. No.: US 2019/0042700 A1
(43) Pub. Date: Feb. 7, 2019

(54) APPARATUS AND METHOD FOR REAL-TIME ASSESSMENT, MAPPING, AND BUILDING DATABASES OF QUALITY OF LIFE INDICATORS

(71) Applicant: Imam Abdulrahman Bin Faisal University, Dammam (SA)

(72) Inventor: Wejdan Munawir Mutaib ALOTAIBI, Dammam (SA)

(73) Assignee: Imam Abdulrahman Bin Faisal University, Dammam (SA)

(21) Appl. No.: 15/667,154

(22) Filed: Aug. 2, 2017

(51) Int. Cl. G06F 19/00; A61B 5/00

(52) U.S. Cl. CPC G06F 19/3425; G06F 19/324; G06F 19/363; A61B 5/0022

(57) ABSTRACT

An apparatus and method for real-time assessment and mapping of quality of life indicators includes: selecting a set of quality of life dimensions, receiving objective quality of life indicators based on the quality of life dimensions, receiving subjective quality of life indicators based on the quality of life dimensions, performing a statistical analysis on the objective quality of life indicators and the subjective quality of life indicators, storing the objective quality of life indicators, the subjective quality of life indicators, and the results of the statistical analysis in a database, and outputting the objective quality of life indicators, the subjective quality of life indicators, and the results of the statistical analysis.

### BACKGROUND OF THE INVENTION

Quality of Life has been employed in a wide range of fields, including national and international development, healthcare, pharmaceutical clinical trials, politics and employment. In some realms, Quality of Life (and the closely related ideas of “well-being” and “happiness”) has replaced GDP and other purely economic or material metrics as a more humane and spiritual measure of the state of a nation. For example, in the Kingdom of Bhutan, Gross National Happiness (GNH) policy guides its processes for economic and development planning. In Bhutan, a development plan must pass a GNH review that is similar to an Environmental Impact Statement required for development in the US. And in July 2011, the United Nations passed Resolution 65/309 (adopted unanimously by the General Assembly) that “invites Member States to pursue the elaboration of additional measures that better capture the importance of the pursuit of happiness and well-being in development with a view to guiding their public policies.”

It is clear that Quality of Life is both an important concept and a useful gauge of individual, and a population's, well-being. The experience of well-being may change over time as a result of external influences, such as, age, maturity, illness, life experience, employment, peer influences, as well as other social, economic, and political situations. But on a national or local level, Quality of Life may be used to quantify the general state of affairs and, more importantly, to measure the efficacy of products, policies, and programs targeted at improving the lives of individuals and groups.

Known methods of Quality of Life assessment involve significant time and effort, involving hundreds or thousands of researchers and data workers to gather and process data over many weeks or months. By the time a Quality of Life assessment is complete, conditions may well have changed significantly.

The “background” description provided herein is for the purpose of generally presenting the context of the disclosure. Work of the presently named inventors, to the extent it is described in this background section, as well as aspects of the description which may not otherwise qualify as conventional art at the time of filing, are neither expressly nor impliedly admitted as conventional art against the present disclosure.

### SUMMARY

Real-time assessment and mapping of Quality of Life indicators may be used to guide the development of products, policies and programs of governments, industries, organizations, researchers, business executives, politicians, and policy-makers to understand the history, trends and current state of Quality of Life, plan intervention strategies, and implement other changes to improve the overall Quality of Life.

An apparatus and method for real-time assessment and mapping of quality of life indicators is disclosed that includes: selecting a set of quality of life dimensions, receiving objective quality of life indicators based on the quality of life dimensions, receiving subjective quality of life indicators based on the quality of life dimensions, performing a statistical analysis on the objective quality of life indicators and the subjective quality of life indicators, storing the objective quality of life indicators, the subjective quality of life indicators, and statistical analysis in a database, and outputting the objective quality of life indicators, the subjective quality of life indicators, and the results of the statistical analysis.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1 is a block diagram illustrating an overall environment in which an example implementation may be practiced;

FIG. 2 is a flow diagram illustrating an example implementation;

FIG. 3 is a flow diagram illustrating another example implementation:

FIG. 4 is an output map according to one example; and

FIG. 5 is a block diagram illustrating a structure of a real-time Quality of Life server.

### DETAILED DESCRIPTION

Quality of Life is a widely used term to capture and quantify the general well-being of individuals and societies. One group of researchers defines Quality of Life as: “an overall general wellbeing that comprises objective descriptors and subjective evaluations of physical, material, social, and emotional wellbeing together with the extent of personal development and purposeful activity, all weighted by a personal set of values” (Felce, D., & Perry, J. “Quality of Life: Its definition and measurement research,” Research in Developmental Disabilities, 16, 51-74, 1995). Another group of researchers provide the following insight: “QOL is the extent to which objective human needs are fulfilled in relation to personal or group perceptions of subjective well-being. Human needs are basic needs for subsistence, reproduction, security, affection, understanding, participation, leisure, spirituality, creativity/emotional expression, identity, freedom. Subjective well-being is assessed by responses to questions about happiness, life satisfaction, utility, or welfare, and the relation between specific human needs and perceived satisfaction is influenced by mental capacity, cultural context, information, education, temperament. In addition, the relation between the fulfillment of human needs and overall subjective well-being is affected by the weights that individuals, groups, and cultures give to fulfilling each of the human needs relative to the others.” (Costanza, R., Fisher, B., Ali, S., Beer, C., Bond, L., Boumans, R., & Snapp, R. (2007). Quality of Life: An approach integrating opportunities, human needs, and subjective well-being. Ecological Economics, 61, 267-276.)

Although there is no universally recognized standard for measuring Quality of Life, there is general agreement that it is a complex, culturally informed, multidimensional notion encompassing both subjective and objective indicators. Subjective indicators may include (but are not limited to): life satisfaction, sense of optimism, sense of security/safety, personal expectations, social relationships, cultural perspectives, interpretation of facts and events, values, religious beliefs, and level of acceptance of current conditions. Objective indicators of Quality of Life may include (but are not limited to): physical health, mental health, level of independence, family, community & friends (social connections), education, wealth/income, income distribution and inequality, employment and work situation, local services, housing, and environment.

Referring now to the drawings, wherein like reference numerals designate identical or corresponding parts throughout the several views.

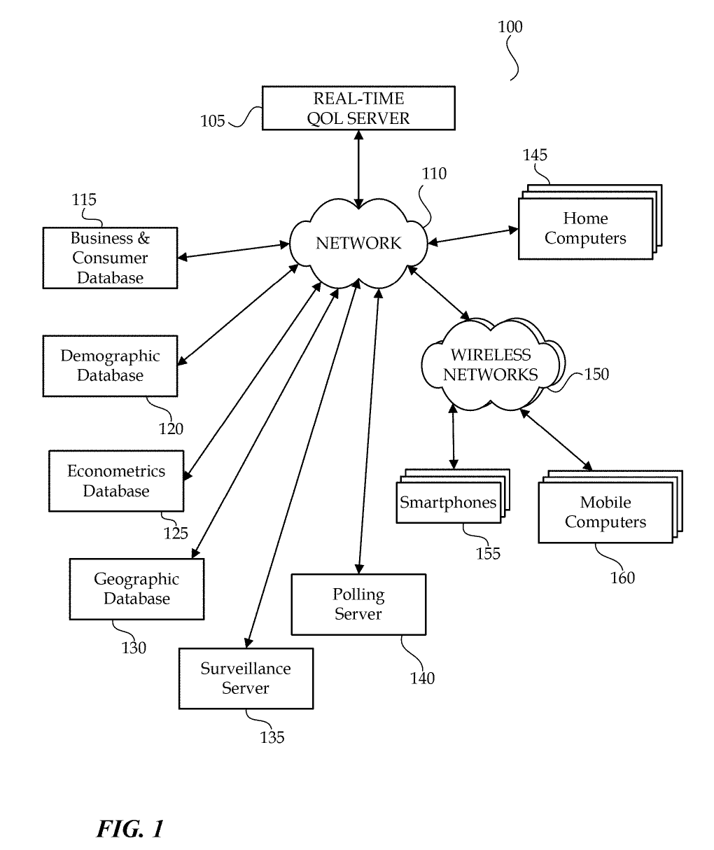

FIG. 1 is a block diagram illustrating an exemplary overall environment 100 in which an embodiment of the present invention may be practiced. Referring to FIG. 1, Real-Time Quality of Life Server 105 includes circuitry and software to perform real-time processing, including a processor with circuitry capable of communicating over a network 110 (the Internet, for example), storing and accessing data, processing data, and presenting data in real time on a display or in printed form. Real-Time Quality of Life Server 105 also includes at least one non-transitory computer readable storage medium that has stored on it at least one computer program to instruct the circuitry to transmit or receive specific data through network 110 in real time, store and access specific data on one or more storage devices within or attached to Real-Time Quality of Life Server 105, process specific data, and present specific data on a display or in printed form. While Real-Time Quality of Life Server 105 is illustrated as a single block in FIG. 1, those skilled in the art will recognize that, without any loss of generality, it may include a plurality of devices that form a distributed real-time processing platform, where the devices, either individually or as a unified whole, includes the capabilities and components listed above with reference to Real-Time Quality of Life Server 105. Real-Time Quality of Life Server 105 may be enhanced through additional hardware and operating system software to perform all necessary processing in real time.

Referring again to FIG. 1, Real-Time Quality of Life Server 105 may communicate over network 110 with a number of database providers and/or other information sources. These may include, but are not limited to, Business and Consumer Database 115, Demographic Database 120, Econometrics Database 125, Geographic Database 130, Surveillance Server 135, and Polling Server 140. The list of database providers and other information servers is exemplary and not intended to be exhaustive. In addition, Real-Time Quality of Life Server 105 may communicate over network 110 with a plurality of users (individuals in a group or population to be assessed) through one or more Home Computers 145, or, through one or more Wireless Networks 150, to one or more Smartphones 155, or one or more Mobile Computers 160. The examples of user equipment (Home Computers, Smartphones, Mobile Computers) and connectivity is typical at the time of filing of the present application, but one skilled in the art would recognize that other forms of interaction with individual users are possible and well within the scope of the present disclosure.

In some embodiments, a computer program stored on a non-transitory computer readable storage medium in Real-Time Quality of Life Server 105 may request business and/or consumer data from Business and Consumer Database 115, demographic data from Demographic Database 120, econometric data from Econometrics Database 125, geographic data from Geographic Database 130, surveillance data from Surveillance Server 135, and polling results from Polling Server 140. Each of these data sources may be selected or tuned to provide timely (real-time) data to ensure that Real-Time Quality of Life Server 105 can provide its Quality of Life information in real-time.

Consumer and business data gathered by Business and Consumer Database 115 may include, for example, consumer spending levels (essentials, discretionary, etc.), consumer debt burden, income, employment, business revenues, housing starts, sales and profits, and trends for each of these. The data may be gathered by primary market research (surveys, polls, etc.) and by analysis of government and business data (quarterly reports, annual reports, and other public filings). Demographic data gathered by Demographic Database 120 may include, for example, births, deaths, marital status, literacy/education, home ownership, property values, crime statistics, etc. Demographic data gathered may be gathered from government statistics, news agencies, and other public and private research. Econometric data gathered by Econometrics Database 125 may include, for example, productivity, per capita GDP, financial market levels and trends, etc. Econometric data may be gathered from government statistics and other public and private research. Geographic data gathered by Geographic Database 130 may include, for example, land use (and trends), population density, weather, natural disasters and their impacts, etc. Numerous sources of geographic data are available, including government agencies, research institutions, and other public and private data sources.

Each of the databases discussed above may obtain its data though contractual agreements with government and/or private institutions. Database providers may also use web crawling software to search key information web sites and search government/public servers for public records. Most importantly, this data is updated automatically and frequently to assure the accuracy of the real-time assessment of Quality of Life indicators.

In some embodiments, Polling Server 140 may request, on behalf of Real-Time Quality of Life Server 105, the timely completion of surveys through Network 110 and Home Computers 145, Smartphones 155, or Mobile Computers 160. This may be done through email, a web site, or through a custom designed software application (program, app or mobile app) that runs on the Home Computers 145, Smartphones 155, or Mobile Computers 160. The use of mobile apps may help ensure that Real-Time Quality of Life Server 105 can provide up-to-the-minute Quality of Life information. Polling Server 140 may also store and distribute results of polling data that has been collected by means other than through Network 110. For example, polling data may be collected through conventional mail, telephone calls, door-to-door canvassing, focus groups, or other direct or indirect contact with individuals. Polling data may include, for example, satisfaction level with a current life situation (family, health, financial situation, community, government, employment, leisure, crime, safety and security, access to services, food, transportation, etc.), optimism/pessimism about the future, general happiness, health status, importance of key aspects of current life situation, and general sense of well-being.

As discussed above, polling data may also be collected by Polling Server 140 through the use of a computer program or mobile app delivered to one or more of the Home Computers 145, Smartphones 155, or Mobile Computers 160. The mobile app may include a survey to assess conditions that relate to subjective quality of life indicators as discussed above. The mobile app survey may be completed by one or more users of the Home Computers 145, Smartphones 155, or Mobile Computers 160. Once completed, the raw data from the survey may be sent to Polling Server 140. Polling Server 140 may then process the raw data to derive subjective quality of life indicators. Geographic information (GPS data, for example) may also be sent by Home Computers 145, Smartphones 155, or Mobile Computers 160 along with the survey raw data to facilitate geographic segmentation of the subjective quality of life indicators. Individual users may be incentivized to complete the Quality of Life survey by direct payment, coupons, or other methods well known in the art.

In some embodiments, Surveillance Server 135 may use one or more surveillance systems to collect real-time data. This data may include (but is not limited to) still images, video, and/or sound of selected surveillance areas. The surveillance systems may include cameras mounted in and around public places, within public buildings, on manned and unmanned terrestrial vehicles, on earth-orbiting satellites, and on manned and unmanned aerial vehicles (drones). The images, video, and/or sound collected by the surveillance systems may be further processed to measure selected characteristics of the surveillance areas. These characteristics may include population density, size and condition of structures, citizen activity levels, crime, and other features of a community that may reflect Quality of Life within a community. Surveillance Server 135 may also collect real-time “telemetry” data that is indicative of Quality of Life. Telemetry data may include details about systems within individual homes and businesses. This data may include items from appliances and equipment like refrigerators, washers, home entertainment systems, thermostats, smoke detectors, heating and air conditioning units, home and business security systems, and the like, as these items become networked as part of the so-called “Internet of Things” (loT). Although the telemetry data may be consolidated to mitigate privacy concerns, it may provide valuable objective indicators of Quality of Life. For example, home thermostat settings and heating and air conditioning use, along with outdoor temperatures, may indicate general comfort of individuals. As a further example, refrigerator use, and identification of products stored and used, may also be correlated with Quality of Life.

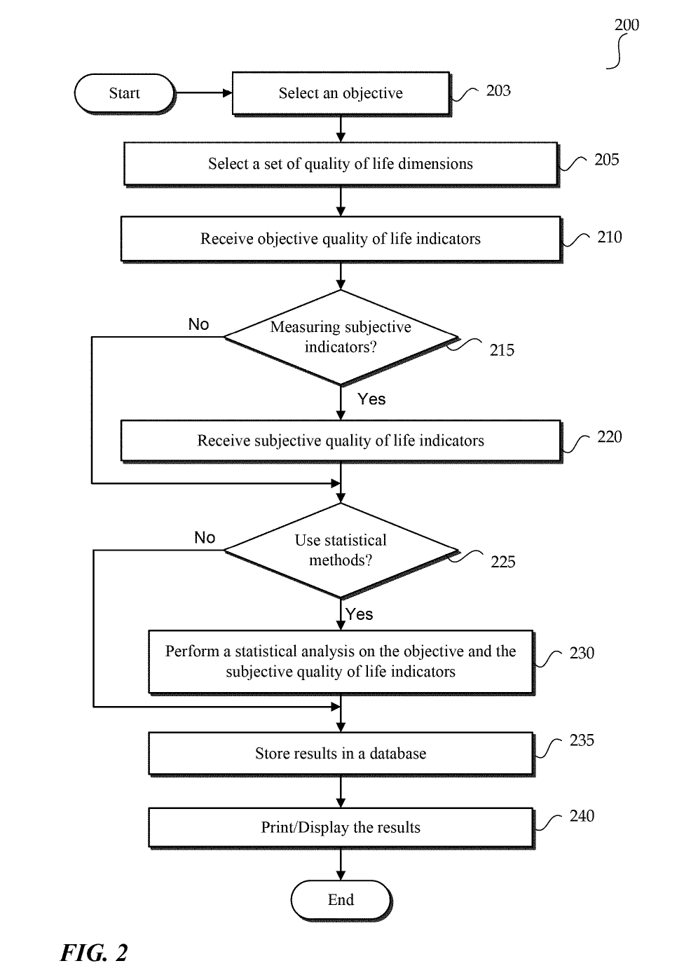

FIG. 2 is a flow diagram illustrating an embodiment of the present invention. The steps illustrated in FIG. 2 are intended to show only a summary of each step of a real-time assessment and mapping process 200. Assessment and mapping process 200 may be part of a computer program stored on a non-transitory computer readable storage medium in or attached to Real-Time Quality of Life Server 105 and executed on Real-Time Quality of Life Server 105.

In step 203 of assessment and mapping process 200, Real-Time Quality of Life Server 105 selects an objective for the Quality of Life assessment and mapping process. The objective may be selected from a group of predetermined Quality of Life objectives stored in a database in or attached to Real-Time Quality of Life Server 105. Each of the predetermined Quality of Life objectives lays out parameters of the assessment and mapping process 200. For example, parameters may include: type of assessment (health, biomedical, political, economic, socioeconomic, etc.), target population, size of assessment group, assessment locale, time frame, etc. The collection of predetermined Quality of Life objectives may be updated from time to time as necessary to achieve optimal results for real-time Quality of Life assessment.

In step 205 of assessment and mapping process 200, Real-Time Quality of Life Server 105 selects a set of Quality of Life dimensions. These dimensions (sometimes referred to as “domains”) may encompass elements that can be measured through a set of specific measurable, quantifiable or computable indicators. The Quality of Life dimensions may include both objective factors (quantifiable from measurable external sources) and subjective factors derived and computed from surveys and polls of individuals or groups by Real-Time Quality of Life Server 105. The number of dimensions may vary depending on the specific objectives of the assessment process. In some embodiments, the number of dimensions may be in a range from 4-8. For example, Schalock lists eight dimensions for Quality of Life: Physical well-being, Material well-being, Social inclusion, Emotional well-being, Rights (civic well-being), Inter-personal relations, Personal development, and Self-determination (Schalock, Robert L., “Three Decades of Quality of Life,” published in Focus on Autism and Other Developmental Disabilities, 15(2):116-127, May 2000). The selection of the appropriate set of Quality of Life dimensions may be dependent, as mentioned above, on the specific objectives of the assessment process. For example, if the assessment process is intended to measure the Quality of Life aspect in a pharmaceutical clinical study, Real-Time Quality of Life Server 105 may select health-related dimensions (physical well-being, emotional well-being, etc.). If, on the other hand, the assessment process is directed toward a more general Quality of Life of a community or other population, the set of dimensions may expand to include other aspects, including material well-being, social inclusion, civic well-being, inter-personal relations, personal development, environmental and economical factors, and self-determination.

In step 210 of assessment and mapping process 200, Real-Time Quality of Life Server 105 receives objective Quality of Life indicators for the target population. The objective Quality of Life indicators may include business and/or consumer data from Business and Consumer Database 115, demographic data from Demographic Database 120, econometric data from Econometrics Database 125, geographic data from Geographic Database 130, and surveillance data from Surveillance Server 135. As discussed above, consumer and business data received may include consumer spending levels, consumer debt burden, income, employment, business revenues, housing starts, sales and profits; demographic data received may include births, deaths, marital status, literacy/education, home ownership, property values, crime statistics; econometric data received may include productivity, per capita GDP, financial market levels and trends, geographic data received may include land use, population density, natural disasters and their impacts; and surveillance data may include population density, size and condition of structures, citizen activity levels, and crime. Those skilled in the art will recognize that other objective Quality of Life indicators may be received from additional sources not shown in FIG. 1. Since the measurement of Quality of Life is a dynamic and evolving process, additional information which may either not be widely available at the time of this writing, or may not have been fully recognized as important to the measurement of Quality of Life may be available to systems that implement the present invention. Receiving such additional information, when and if available, is fully within the spirit of the present disclosure. If the objective of the assessment and mapping process is to construct a geographic map of the target population, then the Quality of Life indicators received during assessment and mapping process 200 can be broken down into geographic segments (states, cities, districts, regions, etc.). In addition, building databases serves researchers and planners.

In step 215 of assessment and mapping process 200, Real-Time Quality of Life Server 105 determines whether subjective Quality of Life indicators will be used. This decision may be based on availability of such subjective data for the selected objective, or may be based on a policy to use only objective measurements. In some assessments, for example, a government agency or business may wish to use strictly objective Quality of Life indicators to avoid a geographic or temporal bias that may result from the use of subjective Quality of Life indicators. In other situations (for example, before a substantial government and/or private investment in urban renewal), a larger and more intimate picture of the general public may be preferred, so the use of subjective Quality of Life indicators may be chosen.

If the decision is made to use subjective Quality of Life indicators, then the assessment and mapping process 200 continues with step 220 where Real-Time Quality of Life Server 105 receives subjective Quality of Life indicators. The subjective Quality of Life indicators may be obtained using polling data from Polling Server 140, or from other sources, either internal or external to Real-Time Quality of Life Server 105. As discussed above, the polling data from Polling Server 140 may be obtained in many different ways from individuals or groups. Again, if the objective of the assessment and mapping process is to construct a geographical map of the target population, then the subjective Quality of Life indicators can be broken down into geographical segments (states, cities, districts, regions, etc.). Further, built databases may be used by researchers and planners.

If the decision is made not to use subjective Quality of Life indicators, step 220 may be skipped.

In step 225 of assessment and mapping process 200, Real-Time Quality of Life Server 105 determines whether to use statistical methods to process the received Quality of Life indicators. The decision whether to use statistical methods may be based on whether or not the end result is to be viewed as raw data or heavily processed and distilled, perhaps into a single Quality of Life level for each individual or group of individuals. In general, statistical methods will be preferred to reduce the bulk raw data into concise and comprehensible results.

If the determination is made to use statistical methods, then the assessment and mapping process 200 continues with step 230 where Real-Time Quality of Life Server 105 performs statistical analysis of the received Quality of Life indicators.

In an embodiment, the statistical analysis of the Quality of Life indicators may use a weighted sum of the individual indicators. For example, if there are n Quality of Life indicators, each represented by a normalized scaler quantity Qᵢ, where i is in the range [1,n] the weighted sum QOL (the Quality of Life level) may be computed from equation (1).

QOL=Σᵢ₌₁ⁿwᵢQᵢ (1)

where wᵢ is a weighting factor for the i^{th} Quality of Life indicator.

Determination of the weighting factors wᵢ may be accomplished through an analysis of the individual indicators (it may be established through extensive testing or polling that, for example, health factors and sense of security are more important to Quality of Life than, for example, physical environment or education) and through an iterative process where the weighting factors are adjusted to optimize the correlation between measured Quality of Life and the results of additional surveys and observations by field researchers. In some embodiments, the weighting factors may be determined by using a correlational statistical method such as Principal Component Analysis or Factor Analysis. Using Factor Analysis, for example, the weighting factors may be determined by computing a correlation matrix for the received Quality of Life indicators, and finding the weighting factors that provide a best fit to the data. In factor analysis, the best fit is defined as the minimum of the mean square error in the off-diagonal residuals of the correlation matrix.

In a simplified example, three objective quality of life indicators Q₁, Q₂, and Q₃ (representing, for example, consumer spending levels, business revenues, and crime statistics respectively) and three subjective quality of life indicators Q₄, Q₅, and Q₆ (representing, for example, life satisfaction, sense of optimism, and sense of security/safety respectively) may be received by Real-Time Quality of Life Server 105. All of the indicators may be geographically consolidated (for each region in a targeted area, for example) and normalized by Real-Time Quality of Life Server 105, for example, to a number in the range of 0 to 10, where 10 indicates a value that is correlated to a high quality of life level and 0 indicates a value that is correlated to a low quality of life level. In addition, in this example, an initial set of weighting factors may be used by Real-Time Quality of Life Server 105 based on preexisting research that identifies the contribution of each of the selected quality of life indicators to an overall quality of life level. For example, the initial set of weighting factors w₁ to w₆ for the six selected indicators Q₁ to Q₆ may be 0.15, 0.07, 0.22, 0.28, 0.17, and 0.11. The Quality of Life level, QOL, may be computed using equation 1 as:

QOL=0.15Q₁+0.07Q₂+0.22Q₃+0.28Q₄+0.17Q₅+0.11Q₆ (2)

If the values received by Real-Time Quality of Life Server 105 for the six selected indicators Q₁ to Q₆, consolidated and normalized for a particular geographic region, are 6, 7, 3, 7, 5, 5, then the Quality of Life level, QOL=5.41.

Those skilled in the art will recognize that other statistical methods may be used to process the quality of life indicators to yield a Quality of Life level. Other methods may also be used to refine the parameters (weighting factors, for example) used for the statistical analysis, including iterative and genetic computational techniques to refine these parameters over the course of one or more Quality of Life assessments.

Referring again to FIG. 2, after the statistical analysis of step 230, the assessment and mapping process 200 continues to step 235, where the Real-Time Quality of Life Server 105 stores results in a database. In this step, some or all of the raw data collected may be stored in a database associated with Real-Time Quality of Life Server 105. In addition, the processed results of the statistical analysis (Quality of Life level for each geographic region or each group of people surveyed, for example) may also be stored in a database associated with Real-Time Quality of Life Server 105. The processed results of the statistical analysis may be stored in a multiple formats. In addition, the stored processed results may be accessed by multiple software applications (e.g., ArcGIS™, ERDAS™, ENVI®, Google Earth™)

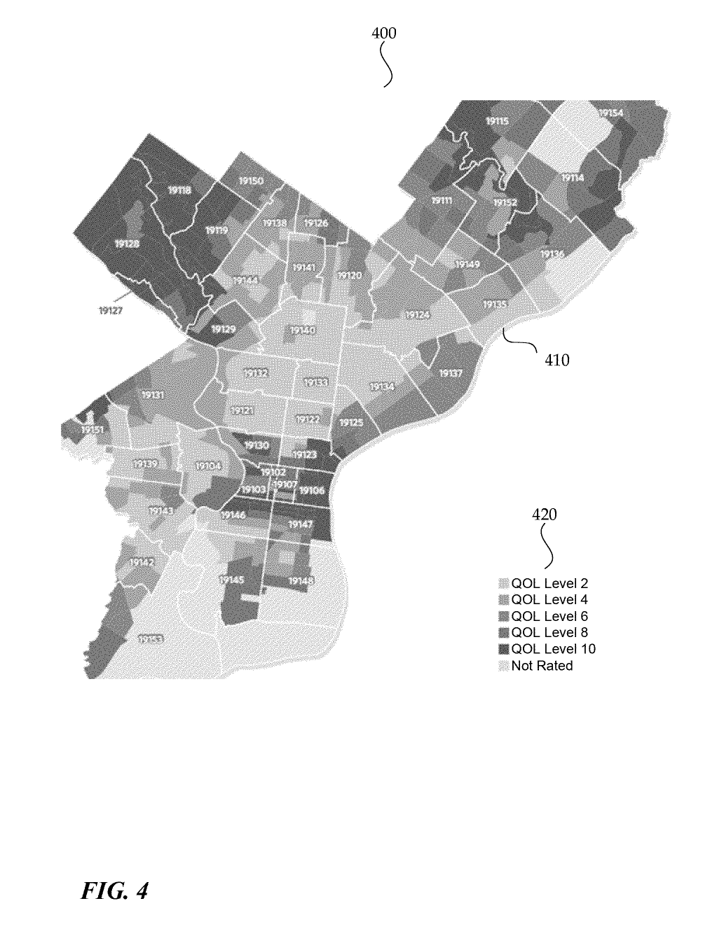

In step 240 of the assessment and mapping process 200, Real-Time Quality of Life Server 105 may print and/or display the results to a user connected to Real-Time Quality of Life Server 105. The results, in some embodiments, may be formatted into the form of a map where areas of a city, for example, may be illustrated with shading or color to show particular Quality of Life levels. FIG. 4 illustrates an output map according to an embodiment of the present invention. The output map may be printed or may be displayed on a user screen. Referring to FIG. 4, output map 400 comprises a shaded city map 410 and legend 420. The shaded city map 410 may employ grayscale shading, as shown, or alternatively use patterns or colors to indicate different Quality of Life levels. Legend 420 may indicate the Quality of Life level for each of the shaded, patterned or colored areas.

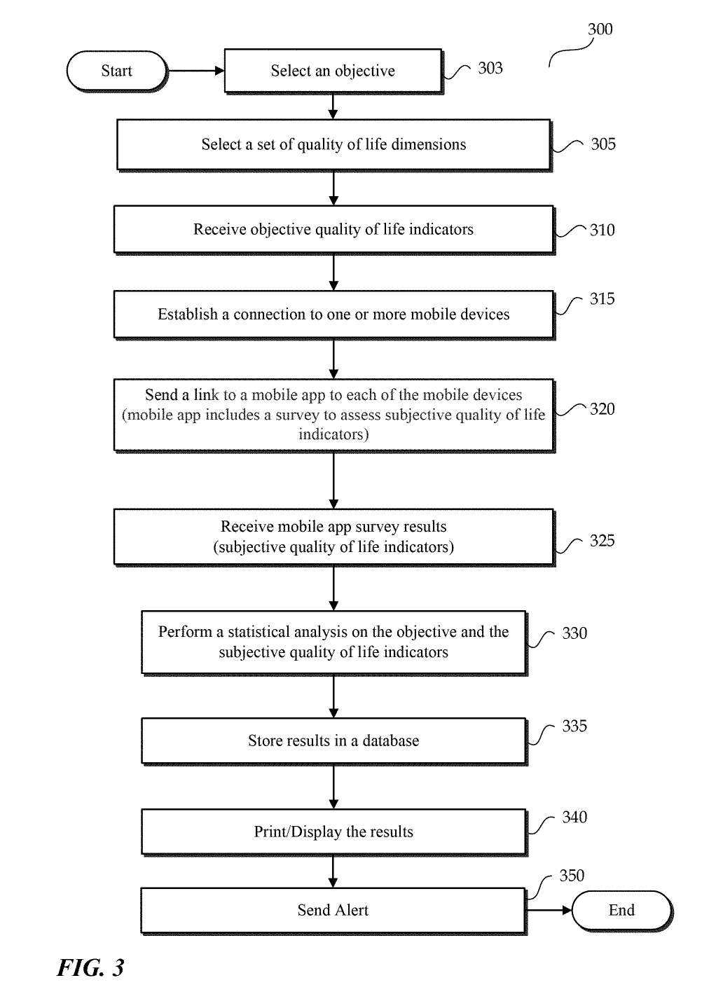

FIG. 3 shows a flow diagram illustrating another implementation. The steps illustrated in FIG. 3, as was the case for the embodiment illustrated in FIG. 2, are intended to show only a summary of each step of an assessment and mapping process 300. Assessment and mapping process 300 may be part of a computer program stored on a non-transitory computer readable storage medium in or attached to Real-Time Quality of Life Server 105 and executed on Real-Time Quality of Life Server 105.

In step 303 of assessment and mapping process 300, Real-Time Quality of Life Server 105 selects an objective for the Quality of Life assessment and mapping process. As noted above with reference to FIG. 2, an objective may be selected from a group of predetermined Quality of Life objectives. Each of the predetermined Quality of Life objectives lays out parameters of the assessment and mapping process 300. For example, parameters may include: type of assessment (health, biomedical, political, economic, socioeconomic, etc.), target population, size of assessment group, assessment locale, time frame, etc. The collection of predetermined Quality of Life objectives may be updated from time to time as necessary to achieve optimal results for real-time Quality of Life assessment.

In step 305 of assessment and mapping process 300, Real-Time Quality of Life Server 105 selects a set of Quality of Life dimensions. As in the assessment and mapping process described above with reference to FIG. 2, these dimensions may encompass elements that can be measured through a set of specific measurable, quantifiable or computable indicators. The Quality of Life dimensions may include both objective factors (quantifiable from measurable external sources) and subjective factors derived and computed from surveys and polls of individuals or groups by Real-Time Quality of Life Server 105. The number of dimensions may vary depending on the specific objectives of the assessment process. In some embodiments, the number of dimensions may be in a range from 4-8. For example, Schalock lists eight dimensions for Quality of Life: Physical well-being, Material well-being, Social inclusion, Emotional well-being, Rights (civic well-being), Inter-personal relations, Personal development, and Self-determination (Schalock, Robert L., “Three Decades of Quality of Life,” published in Focus on Autism and Other Developmental Disabilities, 15(2):116-127, May 2000). The selection of the appropriate set of Quality of Life dimensions may be dependent, as mentioned above, on the specific objectives of the assessment process. For example, if the assessment process is intended to measure the Quality of Life aspect in a pharmaceutical clinical study, Real-Time Quality of Life Server 105 may select health-related dimensions (physical well-being, emotional well-being, etc.). If, on the other hand, the assessment process is directed toward a more general Quality of Life of a community or other population, the set of dimensions may expand to include other aspects, including material well-being, social inclusion, civic well-being, inter-personal relations, personal development, and self-determination.

In step 310 of assessment and mapping process 300, Real-Time Quality of Life Server 105 receives objective Quality of Life indicators for a target population. The objective Quality of Life indicators as it was for the embodiment illustrated in FIG. 2, may include, but is not limited to, data from the sources illustrated in FIG. 1. And as was the case for the embodiment illustrated in FIG. 2, the objective Quality of Life indicators received during assessment and mapping process 300 may be broken down into geographic segments.

In step 315 of assessment and mapping process 300, Real-Time Quality of Life Server 105 establishes a data connection to one or more mobile devices (smartphones 155 and mobile computers 160 as illustrated in FIG. 1, for example) through, for example, network 110 and/or cellular network 150 as illustrated in FIG. 1.

In step 320 of assessment and mapping process 300, of assessment and mapping process 300, Real-Time Quality of Life Server 105 sends a link (or causes a link to be sent) for a mobile app (sometimes called a “mobile application” or simply an “app”) to each of the mobile devices connected in step 315. The mobile app may be associated with Polling Server 140. The mobile app may include a survey to assess conditions that relate to subjective quality of life indicators. The mobile app survey may be completed by one or more users of the mobile devices. Once completed, the raw data from the survey may be sent to Polling Server 140. Polling Server 140 may then process the raw data to derive subjective quality of life indicators. Geographic information (GPS data, for example) may also be sent by the mobile devices along with the survey raw data to facilitate geographic segmentation of the subjective quality of life indicators. Users may be incentivized to complete the Quality of Life survey by direct payment, coupons, or other methods well known in the art.

The assessment and mapping process 300 continues with step 325—receive mobile app survey results (subjective Quality of Life indicators). The subjective Quality of Life indicators derived from the mobile app surveys may be combined with data from other sources. Again, if the objective of the assessment and mapping process is to construct a geographic map of the target population, then the subjective Quality of Life indicators can be broken down into geographic segments. In addition, spatial and non-spatial databases may be created.

In step 330 of assessment and mapping process 300, Real-Time Quality of Life Server 105 may perform statistical analysis of the objective and the subjective Quality of Life indicators (collectively, the “Quality of Life indicators”) as discussed above with reference to the embodiment illustrated in FIG. 2. Additional subjective quality of life indicators may be added for the program.

In step 335 of assessment and mapping process 300, Real-Time Quality of Life Server 105 stores results in a database. In this step, some or all of the raw data collected may be stored in a database associated with Real-Time Quality of Life Server 105. In addition, the processed results of any statistical analysis may also be stored in a database associated with Real-Time Quality of Life Server 105.

In step 340 of assessment and mapping process 300, Real-Time Quality of Life Server 105 may print and/or display the results to a user connected to the Real-Time Quality of Life Server 105 in the same manner as described above with reference to the embodiment illustrated in FIG. 2.

In step 350 of assessment and mapping process 300, Real-Time Quality of Life Server 105 may send an alert to a sponsor of a particular Quality of Life assessment. In some embodiments, the alert may indicate a significant change in one or more indicators or in the overall Quality of Life level. The alert may be provided using a specific smartphone mobile app, text message, or other similar real-time notification system.

Assessment and mapping process 300, as illustrated in FIG. 3, may be used, for example, as part of a pharmaceutical (drug) clinical trial. Clinical trials are used to determine if a drug is both safe and effective for treating a particular condition. Clinical trials in the pharmaceutical industry typically involve the use of either healthy individuals (no medical condition) or patients with specific health conditions who have volunteered to try an experimental drug regimen. In some clinical trials, subjects may remain under supervision for more than 6 weeks. In many clinical trials, in addition to evaluating the physical/medical effects of a new treatment, the subjects' Quality of Life may also be measured. In some embodiments, clinical trial subjects may be provided with a smartphone mobile app or computer program to enable them to provide real-time feedback of subjective quality of life indicators as discussed above with reference to FIGS. 2 and 3. One or more clinical trial sponsors may be provided with a real-time display of the Quality of Life levels of the clinical trial subjects. In addition, alerts may be provided to one or more clinical trial sponsors, in real-time via an application running on a mobile device including automatically opening the application in emergency situations based on the type of alert, should an exceptional situation (sudden change in reported subjective quality of life indicators, for example) occur with one or more of the trial subjects. Such an occurrence may indicate a serious (possibly life threatening) issue with the drug under test. Clinical trial Quality of Life assessment may also use objective indicators from real-time telemetry data as described above. Telemetry data may include details about systems within the homes of clinical trial subjects. As described above, this data may include items from appliances and equipment like refrigerators, washers, home entertainment systems, thermostats, smoke detectors, heating and air conditioning units, home security systems, and the like. For example, home thermostat settings and heating and air conditioning use, along with outdoor temperatures, may indicate general comfort of individuals. As a further example, refrigerator use, and identification of products stored and used, may also be correlated with the Quality of Life of a clinical trial subject.

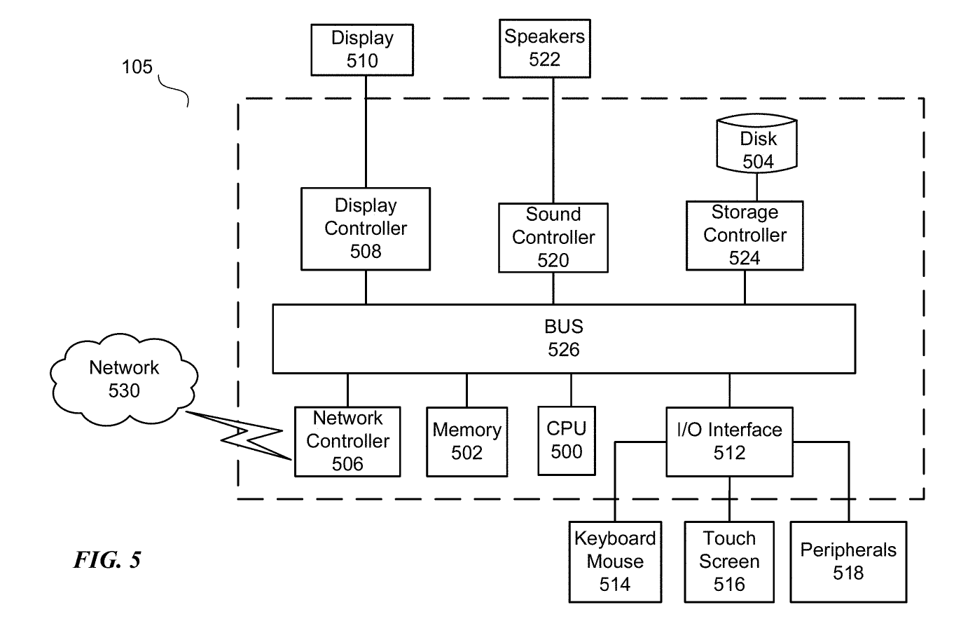

Next, a hardware description of Real-Time Quality of Life Server 105 according to exemplary embodiments is described with reference to FIG. 5. In FIG. 5, Real-Time Quality of Life Server 105 includes a CPU 500 which performs the processes described herein. The process data and instructions may be stored in memory 502. The process data and instructions may also be stored on a storage medium disk 504 such as a hard disk drive (HDD) or solid state drive (SSD). Additional storage media may be connected directly to Real-Time Quality of Life Server 105 through I/O interface 512 or may be connected (locally or remotely) through network controller 506. Further, the claimed advancements are not limited by the form of the computer-readable media on which the instructions of the inventive process are stored. For example, the instructions may be stored on CDs, DVDs, in Flash memory, RAM, ROM, PROM, EPROM, EEPROM, hard disk or any other information processing device with which the Real-Time Quality of Life Server 105 communicates, such as another server or computer. As described previously herein, the processed results may be stored in databases in multiple formats (e.g., KML, SHP).

Further, the claimed advancements may be provided as a utility application, background daemon, or component of an operating system, or combination thereof, executing in conjunction with CPU 500 and an operating system such as Microsoft Windows, UNIX, Solaris, Linux, Apple Mac-OS and other systems known to those skilled in the art.

The hardware elements in order to achieve the Real-Time Quality of Life Server 105 may be realized by various circuitry elements, known to those skilled in the art. For example, CPU 500 may be a Xeon® or Core™ processor from Intel® Corporation or an Opteron™ processor from AMD®, or may be other processor types that would be recognized by one of ordinary skill in the art. Alternatively, the CPU 500 may be implemented on an FPGA, ASIC, PLD or using discrete logic circuits, as one of ordinary skill in the art would recognize. Further, CPU 500 may be implemented as multiple processors, or processors with multiple cores, cooperatively working in parallel to perform the instructions of the inventive processes described herein.

Real-Time Quality of Life Server 105 also includes a network controller 506, such as an Intel® Ethernet PRO network interface card, for interfacing with network 530. As can be appreciated, network 530 can be a public network, such as the Internet, or a private network such as an LAN or WAN network, or any combination thereof and can also include PSTN or ISDN sub-networks. Network 530 can also be wired, such as an Ethernet network, or can be wireless such as a cellular network including EDGE, 3G and 4G wireless cellular systems. The wireless network can also be WiFi, Bluetooth, or any other wireless form of communication that is known.

General purpose storage controller 524 connects the storage medium disk 504 with bus 526, which may be an ISA, EISA, VESA, PCI, PCI-E, or similar, for interconnecting all of the components of Real-Time Quality of Life Server 105. A description of the general features and functionality of display 510, keyboard and/or mouse 514, as well as display controller 508, storage controller 524, network controller 506, sound controller 520, and general purpose I/O interface 512 is omitted herein for brevity as these features are well known.

The exemplary circuit elements described in the context of the present disclosure may be replaced with other elements and structured differently than the examples provided herein. Moreover, circuitry configured to perform features described herein may be implemented in multiple circuit units (e.g., chips), or the features may be combined in circuitry on a single chipset.

The apparatus and methods described herein deliver a substantial improvement to current methods for Quality of Life assessment. Not only is the system fully automated and streamlined, but the interactions with the population and individual stakeholders is focused on real-time data gathering and results generation such that a truer and timelier picture of the well-being of individuals and/or a population may be produced. In addition, the statistical processing of the data may be successively improved over many iterations of the measurement process.

Obviously, numerous modifications and variations of the present invention are possible in light of the above teachings. It is therefore to be understood that within the scope of the appended claims, the invention may be practiced otherwise than as specifically described herein.

1. An apparatus for real-time assessment and mapping of clinical trial quality of life indicators comprising:
a real-time server operatively connected to a network and including circuitry configured to:
select a set of quality of life dimensions from a predetermined collection:
receive objective quality of life indicators from one or more data sources based on the quality of life dimensions;
receive subjective quality of life indicators from one or more clinical trial subjects based on the quality of life dimensions;
perform a statistical analysis on the objective quality of life indicators and the subjective quality of life indicators to produce a quality of life level for at least one of the one or more clinical trial subjects;
store the objective quality of life indicators, the subjective quality of life indicators, and the quality of life level for the at least one of the one or more clinical trial subjects in a database;
output the objective quality of life indicators, the subjective quality of life indicators, and the quality of life level for the at least one of the one or more clinical trial subjects to a display and/or printer; and
provide an alert, if one or more of the subjective quality of life indicators is outside of a predetermined range, to one or more clinical trial sponsors using a smartphone mobile app and/or text message.

2. The apparatus for real-time assessment and mapping of clinical trial quality of life indicators of claim 1, wherein the quality of life dimensions include physical well-being and emotional well-being.

3. The apparatus for real-time assessment and mapping of clinical trial quality of life indicators of claim 1, wherein the circuitry is further configured to derive at least one of the objective quality of life indicators from data received from one or more home appliances of the one or more clinical trial subjects.

4. The apparatus for real-time assessment and mapping of clinical trial quality of life indicators of claim 3, wherein the one or more home appliances includes a thermostat.

5. The apparatus for real-time assessment and mapping of clinical trial quality of life indicators of claim 1, wherein the circuitry is further configured to derive at least one of the subjective quality of life indicators from data collected by a mobile app.

6. The apparatus for real-time assessment and mapping of clinical trial quality of life indicators of claim 1, wherein the statistical analysis includes a weighted sum of the objective quality of life indicators and the subjective quality of life indicators.

7. The apparatus for real-time assessment and mapping of clinical trial quality of life indicators of claim 6, wherein a set of weighting factors is determined using factor analysis.

8. The apparatus for real-time assessment and mapping of clinical trial quality of life indicators of claim 1, wherein the output is in the form of a map, where a quality of life level in indicated for a plurality of regions of the map.

9. A method for real-time assessment and mapping of clinical trial quality of life indicators comprising:
establishing a connection between a real-time server and one or more mobile devices, wherein each of the one or more mobile devices is used by a clinical trial subject:
outputting, from the server, a link to a mobile application at each of the one or more mobile devices, wherein the mobile app includes a survey to assess subjective quality of life indicators;
receiving, at the server, results of the survey from at least one of the one or more mobile devices;
receiving, at the server, additional quality of life indicators from one or more sources of objective quality of life indicators;
performing, at the server, a statistical analysis on the results of the survey and the additional quality of life indicators;
outputting, from the server, the statistical analysis, the results of the survey, and the additional quality of life indicators to a display and/or printer; and
providing an alert, if one or more of the subjective quality of life indicators is outside of a predetermined range, to one or more clinical trial sponsors using a smartphone mobile app and/or text message.

10. The method for real-time assessment and mapping of clinical trial quality of life indicators of claim 9, wherein the survey includes questions to assess physical well-being and emotional well-being.

11. The method for real-time assessment and mapping of clinical trial quality of life indicators of claim 9, wherein at least one of the objective quality of life indicators is derived from data received from one or more home appliances of the one or more clinical trial subjects.

12. The method for real-time assessment and mapping of clinical trial quality of life indicators of claim 11, wherein the one or more home appliances includes a thermostat.

13. The method for real-time assessment and mapping of clinical trial quality of life indicators of claim 9, wherein at least one of the subjective quality of life indicators is derived from data collected by a mobile app.

14. The method for real-time assessment and mapping of clinical trial quality of life indicators of claim 9, wherein the statistical analysis includes a weighted sum of the objective quality of life indicators and the subjective quality of life indicators.

15. The method for real-time assessment and mapping of clinical trial quality of life indicators of claim 14, wherein a set of weighting factors is determined using factor analysis.

16. The method for real-time assessment and mapping of clinical trial quality of life indicators of claim 9, further comprising:
adding additional subjective quality of life indicators for a program.

17. A non-transitory computer-readable medium storing computer-readable instructions thereon which when executed by a computer cause the computer to perform a method comprising:
selecting a set of quality of life dimensions from a predetermined collection;
receiving objective quality of life indicators from one or more data sources based on the quality of life dimensions;
receiving subjective quality of life indicators from one or more clinical trial subjects based on the quality of life dimensions;
performing a statistical analysis on the objective quality of life indicators and the subjective quality of life indicators to produce a quality of life level for at least one of the one or more clinical trial subjects;
storing the objective quality of life indicators, the subjective quality of life indicators, and the quality of life level for the at least one of the one or more clinical trial subjects in a database;
outputting the objective quality of life indicators, the subjective quality of life indicators, and the quality of life level for the at least one group of people to a display and/or printer; and
providing an alert, if one or more of the subjective quality of life indicators is outside of a predetermined range, to one or more clinical trial sponsors using a smartphone mobile app and/or text message.

18. The non-transitory computer-readable medium according to claim 17 wherein the quality of life dimensions include physical well-being and emotional well-being.

19. The non-transitory computer-readable medium according to claim 17 wherein at least one of the objective quality of life indicators is derived from data received from one or more home appliances of the one or more clinical trial subjects.

20. The non-transitory computer-readable medium according to claim 19 wherein the one or more home appliances is a thermostat.
